(19) United States
(12) Patent Application Publication
(10) Pub. No.: US 2025/0107815 A1
(43) Pub. Date: Apr. 3, 2025

(54) MEDICAL DEVICE

(71) Applicant: TERUMO KABUSHIKI KAISHA, Tokyo (JP)

(72) Inventor: Kosuke NISHIO, Irvine, CA (US)

(73) Assignee: TERUMO KABUSHIKI KAISHA, Tokyo (JP)

(21) Appl. No.: 18/898,863

(22) Filed: Sep. 27, 2024

(30) Foreign Application Priority Data
Sep. 29, 2023 (JP) 2023-169511

(51) Int. Cl. A61B 17/22; A61B 17/00; A61B 17/32

(52) U.S. Cl. CPC A61B 17/22; A61B 17/00234; A61B 17/32; A61B 2017/00238; A61B 2017/00305; A61B 2017/00845; A61B 2017/22041

(57) ABSTRACT

A medical device includes an elongated drive shaft that rotates around a central axis, and an elongated outer tubular shaft disposed outside the drive shaft, in which an outer surface of a distal end of the drive shaft is covered with a distal end covering portion made of resin. The drive shaft is a tubular body in which a plurality of wires or wire rods is arranged and spirally coupled around the axis, and at least a part of the distal end covering portion enters an inside of the drive shaft from between the wire rods.

### CROSS-REFERENCES TO RELATED APPLICATIONS

This application claims priority to Japanese Patent Application No. 2023-169511 filed on Sep. 29, 2023, the entire content of which is incorporated herein by reference.

### TECHNOLOGICAL FIELD

The present invention generally relates to a medical device including a long shaft.

### BACKGROUND DISCUSSION

As a method of treating a lesion such as stenosis or occlusion generated in a blood vessel, there is an endovascular treatment in which treatment is performed from inside the blood vessel using a device percutaneously inserted into the blood vessel. In endovascular treatment, a catheter is used for allowing a medical treatment device to reach a lesion.

For example, a catheter disclosed in U.S. Pat. No. 8,628,549 can cut and remove stenosis such as thrombus, plaque, or a calcified lesion in a blood vessel. The catheter includes a drive shaft made of metal in an outer tube shaft made of metal, and the drive shaft rotates around an axis as a central axis. The outer tube shaft includes a curved portion on a distal end, and an operator can perform an operation to rotate a direction of the outer tube shaft, thereby changing a direction of a cutter provided on the distal end of the outer tube shaft, so that the cutter can be located toward the lesion.

### SUMMARY

When an outer tube shaft includes a curved portion and a rotating drive shaft is disposed therein, the drive shaft bends so as to have the shortest distance in the outer tube shaft, so that this easily comes into contact with an inner surface of the outer tube shaft. Since at least a part of the outer tube shaft and the drive shaft are formed of metal, there is a possibility that durability of the drive shaft decreases and metal powder is generated by friction between them.

The medical device disclosed here is capable of decreasing abrasion of the rotating drive shaft.

A medical device that achieves the above-described object is a medical device including an elongated drive shaft that rotates around an axis in a long axis direction as a central axis, and an elongated outer tubular shaft disposed outside the drive shaft, in which an outer surface of a distal end portion of the drive shaft is covered with a distal end covering portion made of resin.

In the medical device configured as described above, since the distal end portion of the drive shaft is covered with a resin, abrasion when the distal end portion of the drive shaft comes into contact with an inner surface of the outer tubular shaft can be decreased, and the drive shaft can be smoothly rotated.

According to another aspect, a medical device positionable in a biological lumen comprises: an elongated outer tubular shaft configured to be positioned in the biological lumen, with the elongated outer tubular shaft having an inner surface surrounding an interior of the elongated outer tubular shaft; and a rotatable elongated drive shaft positioned in the interior of the elongated outer tubular shaft so that a distal portion of the elongated outer tubular shaft surrounds and axially overlaps at least a distal portion of the rotatable elongated drive shaft. The rotatable elongated drive shaft is rotatable relative to the elongated outer tubular shaft. The elongated outer tubular shaft and the rotatable elongated drive shaft are configured so that during rotation of the rotatable elongated drive shaft relative to the elongated outer tubular shaft, and the distal portion of the rotatable elongated drive shaft contacts the inner surface of the elongated outer tubular shaft. A distal end covering portion is located at and covers the distal portion of the rotatable elongated drive shaft, the distal end covering portion is made of resin, and the distal end covering portion having an outer surface. The distal end covering portion includes a proximal end portion terminating at a proximal end of the distal end covering portion, the distal end covering portion also includes a distal end portion terminating at a distal end of the distal end covering portion, and the rotatable elongated drive shaft extends proximally beyond the proximal end of the distal end covering. The outer surface of the distal end covering portion that is made of resin reduces friction at a time of the contact of the rotatable elongated drive shaft with the elongated outer tubular shaft during rotation of the rotatable elongated drive shaft relative to the elongated outer tubular shaft.

In accordance with another aspect, a method comprises positioning an elongated outer tubular shaft in a biological lumen, wherein the elongated outer tubular shaft has an inner surface surrounding an interior of the elongated outer tubular shaft in which is positioned an elongated drive shaft so that a distal portion of the elongated outer tubular shaft surrounds and axially overlaps at least a distal portion of the elongated drive shaft. The elongated drive shaft includes a distal end covering portion located at and covering the distal portion of the elongated drive shaft, wherein the distal end covering portion is made of resin and has an outer surface, with the distal end covering portion including a proximal end portion terminating at a proximal end of the distal end covering portion. The distal end covering portion also includes a distal end portion terminating at a distal end of the distal end covering portion, and the elongated drive shaft extends proximally beyond the proximal end of the distal end covering portion. The method further includes rotating the elongated drive shaft relative to the elongated outer tubular shaft while the elongated outer tubular shaft is positioned in the biological lumen and while the elongated drive shaft is positioned in the interior of the elongated outer tubular shaft. The rotating of the elongated drive shaft relative to the elongated outer tubular shaft results in the outer surface of the distal end covering portion made of the resin to contact the inner surface of the elongated outer tubular shaft, and the outer surface of the distal end covering portion made of resin reduces friction during the contact of the rotatable elongated drive shaft with the elongated outer tubular shaft while the elongated drive shaft is rotating relative to the elongated outer tubular shaft.

### BRIEF DESCRIPTION OF THE DRAWINGS

FIG. 1 is a front view illustrating a medical device according to an embodiment.

FIG. 2 is a perspective view illustrating a distal end of the medical device as seen through a cutter and a housing unit.

FIG. 3 is a cross-sectional view illustrating the distal end of the medical device.

FIGS. 4A to 4C illustrate the distal end of the medical device in which FIG. 4A is a cross-sectional view taken along the section line 4A-4A in FIG. 3, FIG. 4B is a cross-sectional view taken along the section line 4B-4B in FIG. 3, and FIG. 4C is a cross-sectional view taken along the section line 4C-4C in FIG. 3.

FIG. 5 is a cross-sectional view illustrating a portion on a proximal end side from FIG. 3 of the medical device, a front view of a drive shaft.

FIG. 6 is a cross-sectional view in a state in which the drive shaft and a distal end of a distal end covering portion are in close contact with each other.

FIG. 7 is a cross-sectional view in a state in which the drive shaft and a proximal end of the distal end covering portion are in close contact with each other.

FIG. 8 is a schematic diagram illustrating a state in which cutting is performed by the medical device.

FIG. 9 is a cross-sectional view of the vicinity of a coupling portion between the drive shaft and a shaft portion in a case where an outer tubular shaft does not include a curved portion.

### DETAILED DESCRIPTION

Hereinafter, an embodiment of the medical device disclosed here, representing one example of the new medical device, is described with reference to the drawings. The dimensions of the drawings may be exaggerated and different from actual dimensions for convenience of description in some cases. In the present specification and the drawings, components having substantially the same functional configuration are denoted by the same reference numeral, and a detailed description of such features may not be repeated. In the present specification, a side to be inserted into a lumen is referred to as a “distal end side”, and a side to be operated is referred to as a “proximal end side”.

A medical device 10 according to the embodiment is inserted into a blood vessel and used for treatment of cutting and removing plaque, a calcified lesion or the like in acute lower limb ischemia or deep vein thrombosis. An object to be cut is not especially limited, and may be, for example, atheroma, thrombus or the like. Furthermore, all objects that may be present in a biological lumen may correspond to the object to be cut by the medical device 10.

As illustrated in FIGS. 1 to 3, the medical device 10 is provided with a rotatable rotating structure 11, a housing unit 12 that rotatably houses the rotating structure 11, and a handle 90 operated by an operator. The rotating structure 11 is provided with a drive shaft 20 that transmits rotational force, a shaft portion 30 rotatably supported by the housing unit 12, a cutter 50 that cuts plaque and a calcified lesion, and a protection tube 60 housed in the drive shaft 20. The housing unit 12 is provided with an outer tubular shaft 70 that houses the drive shaft 20 and a bearing 80 that rotatably supports the shaft portion 30.

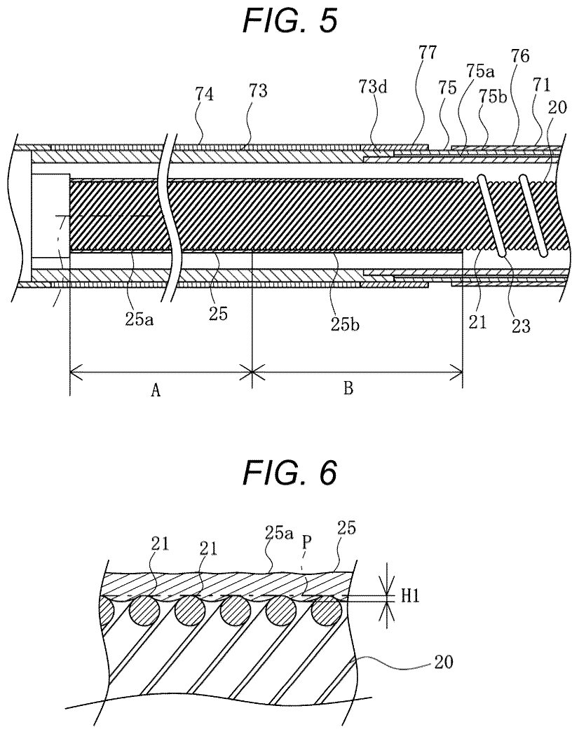

The drive shaft 20 is a long tube body (elongated tubular body). The drive shaft 20 is flexible and has a property capable of transmitting the rotational force applied from a proximal end side to a distal end side. The shaft portion 30 is fixed to a distal end of the drive shaft 20. As illustrated in FIG. 5, the drive shaft 20 is a tubular body in which a plurality of wire rods (wires) 21 is arranged and spirally coupled around an axis X of the rotating structure 11. The drive shaft 20 includes an additional coil 23 spirally wound around an outer peripheral surface thereof from a position on the proximal end side from the distal end toward the proximal end side. The additional coil 23 can generate a flow of fluid that conveys an object toward the proximal end side as the drive shaft 20 rotates. The axis X is a structural central axis of the rotating structure 11, the central axis of the rotating structure 11. A proximal end of the drive shaft 20 is located inside the handle 90. The drive shaft 20 is not necessarily formed of a wire rod.

A constituent material from which the drive shaft 20 may be fabricated is not especially limited as long as the material is metal, and for example, stainless steel, nitinol or the like can be suitably used.

The cutter 50 is a member for cutting the object such as the plaque and calcified lesion into small pieces as illustrated in FIGS. 1 to 3. Therefore, the term “cutting” is intended to mean an action of applying force to an object in contact with the same and making the object smaller. An applying method of force during cutting and a shape or a mode of the cut object are not limited.

The cutter 50 includes a large number of fine abrasive grains on a surface thereof. The cutter 50 may also be provided with a sharp blade. In the cutter 50, a first through-hole 51 located on the distal end side and a second through-hole 52 located on the proximal end side from the first through-hole 51 are formed. The first through-hole 51 and the second through-hole 52 communicate with each other and penetrate the cutter 50 in a direction along the axis X. A distal end of the shaft portion 30 is fitted in the second through-hole 52 to be coupled.

An outer peripheral surface of the cutter 50 includes a groove-shaped cutout 53 extending in the direction along the axis X. The cutout 53 functions as a flow path for conveying the cut object in a proximal end direction. The cutouts 53 are arranged, for example, at intervals of 120 degrees in a circumferential direction. Therefore, the cutter 50 includes three cutouts 53 regularly arranged in the circumferential direction. An edge of each cutout 53 is smoothly formed with a curvature. The number of the cutouts 53 is not limited to three.

It is preferable that a constituent material from which the cutter 50 may be fabricated is strong enough to cut the plaque, calcified lesion or the like, and stainless steel, nitinol, Ta, Ti, Pt, Au, W, brass, a shape memory alloy, a supersteel alloy and the like can be suitably used, for example. In a case where a soft object such as a blood vessel is to be cut, a fluorine-based polymer such as polytetrafluoroethylene (PTFE) and a tetrafluoroethylene-ethylene copolymer (ETFE), polyetheretherketone (PEEK), polyimide, polyolefin such as polyethylene and polypropylene, polyamide, polyester such as polyethylene terephthalate and the like can be suitably used.

The protection tube 60 is a flexible tubular body disposed in the drive shaft 20 and the cutter 50. The protection tube 60 is not fixed to the drive shaft 20 and the cutter 50, and is disposed so as not to rotate together with the drive shaft 20. A guide wire lumen 61 into which a guide wire is inserted is formed in the protection tube 60. The protection tube 60 prevents the guide wire that passes through the inside of the drive shaft 20 from coming into direct contact with and rubbing against the drive shaft 20. A tubular protection tube stopper 62 is coupled to a distal end of the protection tube 60. A proximal end of the protection tube stopper 62 has an outer diameter larger than that of the protection tube 60 and covers the distal end of the protection tube 60. A distal end of the protection tube stopper 62 has an outer diameter smaller than that of the proximal end of the protection tube stopper 62 and protrudes toward the distal end side from the protection tube 60. The proximal end of the protection tube stopper 62 is disposed in a gap between the shaft portion 30 and the cutter 50 in an axial direction. Therefore, the protection tube stopper 62 can restrict movement of the protection tube 60 in the axial direction, and prevent the protection tube 60 from falling.

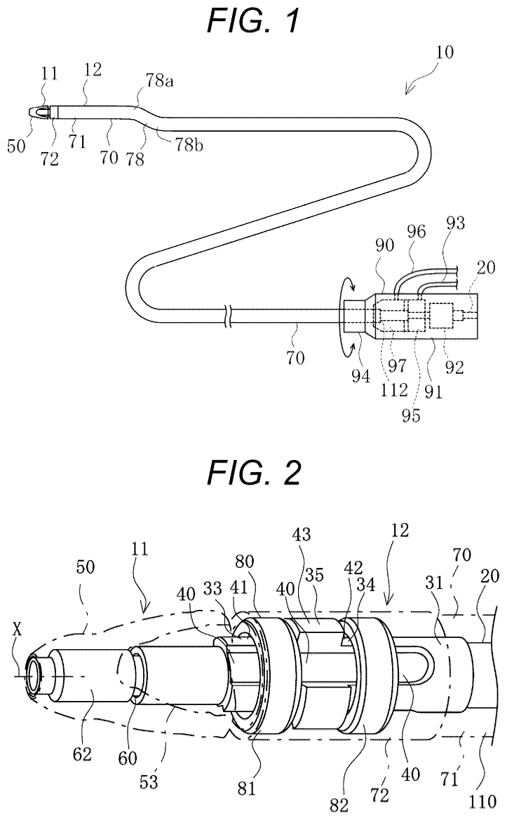

As illustrated in FIGS. 2 to 4, the bearing 80 includes a first bearing 81 and a second bearing 82 each having a ring shape. The first bearing 81 and the second bearing 82 are arranged so as to be spaced apart along the axis X (spaced apart from one another in the axial direction) inside a support tube 72. The first bearing 81 and the second bearing 82 have the same shape, but may have different shapes.

As illustrated in FIGS. 2 to 4, the shaft portion 30 is a site that rotatably supports the rotating structure 11 with respect to the housing unit 12. The shaft portion 30 is provided with a proximal end coupling portion 31 coupled to the drive shaft 20 and a distal end coupling portion 32 coupled to the cutter 50. The shaft portion 30 is further provided with a first sliding portion 33 supported by the first bearing 81, a second sliding portion 34 supported by the second bearing 82, and an intermediate shaft portion 35 disposed between the first sliding portion 33 and the second sliding portion 34. In the shaft portion 30, at least one (three, in the present embodiment) passage 40 extending along the axis X (extending along the axial direction) is formed.

The proximal end coupling portion 31 is disposed on a proximal end of the shaft portion 30 to which the distal end of the drive shaft 20 can be fitted and fixed by, for example, laser welding. The distal end coupling portion 32 is disposed on the distal end of the shaft portion 30 to which the cutter 50 can be fitted and fixed.

The first sliding portion 33 is a site disposed on the proximal end side of the distal end coupling portion 32 and rotatably supported by the first bearing 81. In the first sliding portion 33, three groove-shaped passages 40 extending in the axial direction are formed on an outer peripheral surface of a circular tube having a uniform outer diameter. The first sliding portion 33 includes three first rotary contact portions 41 that come into contact with an inner peripheral surface of the first bearing 81 between the three groove-shaped passages 40 regularly arranged in the circumferential direction. An outer diameter of the first rotary contact portion 41 is smaller than an inner diameter of the first bearing 81 to some extent. Therefore, the first rotary contact portion 41 slidably comes into contact with the inner peripheral surface of the first bearing 81.

The second sliding portion 34 is a site disposed on the distal end side of the proximal end coupling portion 31 and rotatably supported by the second bearing 82. In the second sliding portion 34, three groove-shaped passages 40 extending in the axial direction are formed on an outer peripheral surface of a circular tube having a uniform outer diameter. The second sliding portion 34 includes three second rotary contact portions 42 that come into contact with an inner peripheral surface of the second bearing 82 between the three groove-shaped passages 40 regularly arranged in the circumferential direction. An outer diameter of the second rotary contact portion 42 is smaller than an inner diameter of the second bearing 82 to some extent. Therefore, the second rotary contact portion 42 slidably comes into contact with the inner peripheral surface of the second bearing 82.

The intermediate shaft portion 35 is disposed between (axially between) the first sliding portion 33 and the second sliding portion 34. In the intermediate shaft portion 35, three groove-shaped passages 40 extending along the axis X (axial direction) are formed on an outer peripheral surface of a circular tube having a uniform outer diameter. The intermediate shaft portion 35 includes three protrusions 43 that protrudes radially outward from the first sliding portion 33 and the second sliding portion 34 between the three groove-shaped passages 40 regularly arranged in the circumferential direction.

The passage 40 forms a flow path for taking in the object cut by the cutter 50. A distal end of each passage 40 communicates with a proximal end of the cutout 53. The passage 40 is formed from the distal end side from the bearing 80 to the proximal end side of the bearing 80. The groove-shaped passage 40 may partially penetrate to an inner peripheral surface of the shaft portion 30. The passage 40 is connected to an outer peripheral surface of the shaft portion 30 on the distal end side from the bearing 80. As a result, a space of the passage 40 communicates with a space outside the shaft portion 30 on the distal end side from the bearing 80. The passage 40 is further connected to the outer peripheral surface of the shaft portion 30 on the proximal end side from the bearing 80. As a result, the space of the passage 40 communicates with the space outside the shaft portion 30 on the proximal end side from the bearing 80. Therefore, the passage 40 can take in the cut object from the outside of the shaft portion 30 on the distal end side from the bearing 80. The passage 40 can discharge the object taken in on the distal end side from the bearing 80 to the outside of the shaft portion 30 on the proximal end side from the bearing 80. By providing the passage 40 in the shaft portion 30, an intake opening 111 for taking in a cut piece can be located near the cutter 50. Furthermore, by providing the passage 40 in the shaft portion 30, friction between the shaft portion 30 and the bearing 80 is decreased, and slidability can be improved.

As illustrated in FIGS. 1 to 3, the outer tubular shaft 70 is a tubular body that houses the drive shaft 20 and the protection tube 60. The outer tubular shaft 70 includes an outer tubular main body 71 and a support tube 72 fixed to the distal end side of the outer tubular main body 71. An intake lumen 110 is formed between the outer tubular shaft 70 and the drive shaft 20 for taking in a small object obtained by cutting the plaque, calcified lesion or the like. The outer tubular shaft 70 includes the intake opening 111 on a distal end thereof for taking in the cut object or liquid discharged from the drive shaft 20. The distal end of the outer tubular shaft 70 is disposed with a predetermined gap G from a proximal end of the cutter 50 toward the proximal end side. The gap G has a length exceeding 0 (zero) along the axis X (axial direction) when the rotating structure 11 is disposed on the most proximal end side with respect to the housing unit 12. Therefore, the distal end of the outer tubular shaft 70 is prevented from coming into contact with the cutter 50. The outer tubular shaft 70 includes a proximal end opening 112 that opens inside the handle 90 on a proximal end thereof.

The outer tubular main body 71 is a tubular body having flexibility. The outer tubular main body 71 extends from the handle 90 to the vicinity of the cutter 50. The outer tubular main body 71 includes a curved portion 78 in which an extending direction of the outer tubular main body 71 changes on a distal end thereof. The curved portion 78 includes a first curved portion 78a on the distal end side and a second curved portion 78b on the proximal end side curved in opposite directions, and has an S-shape. The curved portion 78 is not limited to a portion curved twice, and may be curved once or may be curved three or more times. A proximal end of the outer tube main body 71 is fixed to the handle 90. The proximal end opening 112 is disposed on the proximal end of the outer tube main body 71.

The support tube 72 is a circular tube made of metal fixed to the distal end of the outer tubular main body 71. The support tube 72 is provided with a support tubular main body 113 having a certain inner diameter and a stopper 114 disposed on the distal end side of the support tube main body 113 and having an inner diameter smaller than that of the support tubular main body 113. The intake opening 111 is disposed on a distal end of the support tube 72. The stopper 114 comes into contact with a distal end surface of the first bearing 81 of the bearing 80 described later. As a result, the stopper 114 restricts the first bearing 81 from moving toward the distal end side with respect to the support tube 72 and falling from the support tube 72. The stopper 114 can restrict the movement of the first bearing 81 as long as this (the stopper 114) can come into contact with the distal end surface of the first bearing 81, so that this (the stopper 114) may be separated from the first bearing 81 to some extent. The inner diameter of the stopper 114 is preferably smaller than an outer diameter of the first bearing 81 and larger than the inner diameter of the first bearing 81. The structure of the stopper 114 is not especially limited as long as this (the stopper 114) can restrict the movement of the first bearing 81, and for example, the stopper may be partially disposed in the circumferential direction.

As illustrated in FIG. 5, the outer tubular main body 71 includes a distal end tube 73 made of metal and a proximal end tube 75 disposed on the proximal end side from the distal end tube 73. The curved portion 78 of the outer tubular shaft 70 is formed in a range of the distal end tube 73. The proximal end tube 75 includes an inner tube 75a made of resin including a braided body formed of a thin metal wire therein, and an outer tube 75b made of metal that covers an outer surface of the inner tube 75a.

The distal end tube 73 includes a stepped portion 73d on a proximal end of the distal end tube 73. A distal end of the inner tube 75a of the proximal end tube 75 abuts the stepped portion 73d of the distal end tube 73. A proximal end surface of the distal end tube 73 and a distal end surface of the outer tube 75b are butted to each other, and the proximal end of the distal end tube 73 and an outer surface of a distal end of the outer tube 75b are coupled by a coupling member 77 made of metal.

On an outer surface of the distal end tube 73, a region from a proximal end of the support tube 72 to a distal end of the coupling member 77 is covered with a covering material 74 made of resin. An outer surface of the outer tube 75b forming the proximal end tube 75 is covered with a proximal end side covering material 76. A distal end position of the proximal end side covering material 76 is located on the proximal end side from a proximal end of the coupling member 77.

The distal end tube 73 and the outer tube 75b of the proximal end tube 75 are formed of metal as described above. It is preferable that a constituent material from which the distal end tube 73 and the outer tube 75b of the proximal end tube 75 may be fabricated is strong to some extent, and stainless steel, nitinol, Ta, Ti, Pt, Au, W, a shape memory alloy and the like can be suitably used, for example.

The covering material 74 is formed of a flexible resin having excellent slidability. The covering material 74 is desirably melted at certain temperature. In the present embodiment, polyolefin is used as the covering material 74. The covering material 74 may be formed of another resin having the above-described property, and for example, a thermoplastic resin such as polyamide or polyurethane can be appropriately combined with a hydrophilic coating or the like.

The proximal end side covering material 76 can be formed of a resin harder than the covering material 74 that covers the distal end tube 73. As such resin, for example, a nylon elastomer, for example, Pebax (registered trademark) can be used. As a result, the rigidity of the outer tubular shaft 70 can be increased, and operability including torque transmission performance can be made excellent. However, the material of the proximal end side covering material 76 may be another resin, and for example, PTFE, PET and the like can be used.

It is preferable that a constituent material from which the support tube 72 may be fabricated is strong to some extent, and for example, stainless steel, nitinol, Ta, Ti, Pt, Au, W, a shape memory alloy, an engineering plastic such as polyether ether ketone (PEEK), and a combination thereof can be suitably used.

As illustrated in FIG. 5, an outer surface of the distal end portion of the drive shaft 20 is covered with a distal end covering portion 25 made of resin. The distal end covering portion 25 continuously extends from a proximal end surface of the shaft portion 30 toward the proximal end side from a coupling portion between the distal end tube 73 and the proximal end tube 75 in the long axis direction (axial direction). The distal end covering portion 25 extends so as to include a range of the curved portion 78 of the outer tubular shaft 70 in the long axis direction. Since the outer tubular shaft 70 includes the S-shaped curved portion 78, the drive shaft 20 easily comes into contact with the first curved portion 78a and the second curved portion 78b, and at that time, the distal end covering portion 25 comes into contact with the inner surface of the outer tubular shaft 70, so that abrasion of the drive shaft 20 can be decreased. The inner tube 75a of the proximal end tube 75 includes a braided body made of metal therein as described above, and there is a case where the braided body is exposed inside on the distal end; however, since the distal end covering portion 25 is also provided at a joint between the distal end tube 72 and the proximal end tube 75, it is possible that the exposed braided body does not come into direct contact with a metal portion of the drive shaft 20, and durability of the drive shaft 20 can be improved.

The proximal end position of the distal end covering portion 25 is located on the distal end side from the distal end position of the additional coil 23 of the drive shaft 20, and is not provided in a range in which the additional coil 23 is disposed. Therefore, it is possible to prevent the flow path through which the fluid including the object flows from being narrowed in the range in which the additional coil 23 is provided.

When the distal end covering portion 25 is fixed to the drive shaft 20, first, the distal end portion of the drive shaft 20 is covered with a tubular distal end covering portion 25 and heated at certain temperature (for example, about 230° C.) for a certain time. As a result, as illustrated in FIG. 6, a surface portion of the distal end covering portion 25 is melted, and a part thereof enters the inside of the drive shaft 20 from between the wire rods 21. That is, as shown in FIG. 6, a part of the surface portion of the distal end covering portion 25 is located between axially adjacent windings of the wires 21 forming the drive shaft 20.

As a result, the distal end covering portion 25 and the drive shaft 20 can be fixed in close contact with each other.

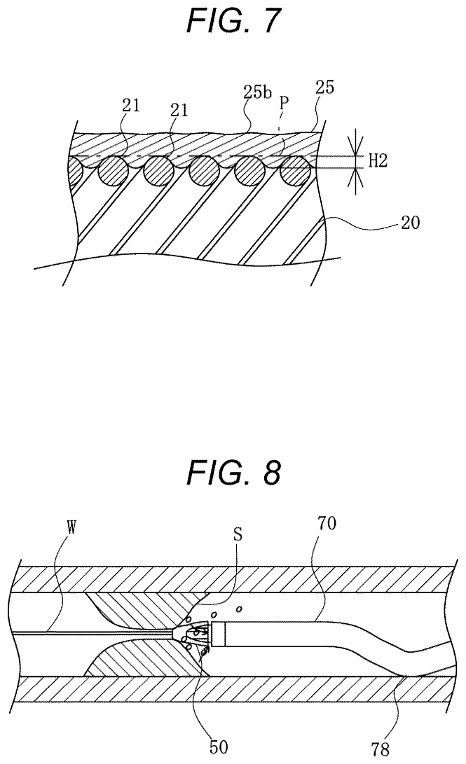

Next, the proximal end 25b of the distal end covering portion 25 is covered with a fluorine-based heat-shrinkable tube that can be removed after shrinkage, and heated at certain temperature (for example, about 230° C.) for a certain time. As a result, as illustrated in FIG. 7, the surface portion of the proximal end 25b of the distal end covering portion 25 melts and enters more deeply the inside of the drive shaft 20 from between the wire rods 21. That is, as shown in FIG. 7, the surface portion of the distal end covering portion 25 is located more deeply between axially adjacent windings of the wires 21 forming the drive shaft 20 so that the surface portion of the distal end covering portion 25 is closer to the central axis. As illustrated in FIG. 5, in the distal end covering portion 25, a range (region A) in which the curved portion 78 of the outer tubular shaft 70 is formed is defined as a distal end 25a, and the proximal end 25b is a range (region B) closer to the proximal end side. The drive shaft 20 is highly likely to be mechanically damaged by the braided body of the inner tube 75a or the like described above in a portion on the proximal end side from the curved portion 78. Since the proximal end 25b of the distal end covering portion 25 enters more deeply the inside of the drive shaft 20 than the distal end 25a, it is possible to prevent peeling or the like of the distal end covering portion 25 in a portion in which the outer tubular shaft 70 is linear on the proximal end side from the curved portion 78.

A depth at which the distal end covering portion 25 enters the inside of the drive shaft 20 (i.e., is positioned inwardly of the outer surface of the wire rods/wires 21), that is, a depth at which the distal end covering portion 25 enters from an imaginary plane P connecting the surfaces of the wire rods (wires) 21 farthest from the axis (axial center) of the wire rods (wires) 21 is more than 0 (zero) μm and 100 μm or less. As illustrated in FIGS. 6 and 7, a depth H2 in the proximal end 25b is larger than a depth H1 in the distal end 25a.

Since the distal end covering portion 25 enters the inside of the drive shaft 20 over the entire length of the distal end covering portion 25, the distal end covering portion 25 continuously enters the inside of the drive shaft 20 from the position on the distal end side from the curved portion 78 to the position on the proximal end side from the curved portion 78. That is, the distal end covering portion 25 is positioned inwardly of the outer surface of the wire rods/wires 21, and enters between the axially adjacent helical windings of the wire rods/wires 21, continuously over the entire length of the distal end covering portion 25, and this distal end covering portion 25 extends continuously from the distal end side of the curved portion 78 to the proximal end side of the curved portion 78. Therefore, friction at the time of contact between the drive shaft 20 and the outer tubular shaft 70 in the curved portion 78 can be reliably decreased. The distal end covering portion 25 does not necessarily continuously cover the distal end portion of the drive shaft 20 in the long axis direction, and may be formed so as to substantially cover a coil-shaped surface portion of the drive shaft 20.

The distal end covering portion 25 is flexible and is formed of a resin that melts at certain temperature. In the present embodiment, polyolefin is used as the distal end covering portion 25. The distal end covering portion 25 may be formed of another resin having the above-described property, and for example, a thermoplastic resin such as polyamide or polyurethane can be appropriately combined with a hydrophilic coating or the like.

As illustrated in FIG. 1, the handle 90 is provided with a casing 91, a drive unit 92, an intake port 93, a rotation operation portion 94, and a liquid delivery port 96.

A proximal end of the outer tubular main body 71 is fixed to a distal end of the casing 91. In the casing 91, an intake space 95 communicating with the intake port 93 and a liquid delivery space 97 communicating with the liquid delivery port 96 are formed. The proximal end opening 112 of the outer tubular main body 71 is rotatably disposed in the intake space 95.

The rotation operation portion 94 is a site operated by an operator with his/her finger to apply rotational torque to the outer tubular shaft 70. The rotation operation portion 94 is rotatably coupled to the distal end of the casing 91. The rotation operation portion 94 is fixed to an outer peripheral surface of the proximal end of the outer tubular main body 71.

The drive unit 92 is, for example, a hollow motor. The drive unit 92 is rotated by a battery not illustrated or externally supplied power. The drive shaft 20 is fixed to a hollow drive rotor of the hollow motor. A rotation speed of the drive unit 92 is not especially limited, and is 5,000 to 200,000 rpm, for example. The configuration of the drive unit 92 is not especially limited.

The intake port 93 conveys an object, liquid or the like in the intake space 95 to the outside. From the liquid delivery port 96, fluid can be delivered to the inside of the outer tubular shaft 70 via the liquid delivery space 97.

Next, a method of using the medical device 10 according to the present embodiment will be described using, as an example, a case where the lesion such as the plaque and calcified lesion in the blood vessel is cut and taken in.

First, the operator inserts a guide wire W into the blood vessel and allows the same to reach the vicinity of a lesion S. Next, the operator inserts a proximal end of the guide wire W into the guide wire lumen 61 of the medical device 10. Thereafter, as illustrated in FIG. 8, the cutter 50 is moved to the vicinity of the lesion S using the guide wire W as a guide. At that time, since the distal end tube 72 forming the distal end of the outer tubular shaft 70 is provided with the covering material 76 made of resin, friction with the blood vessel wall can be controlled to be low. Therefore, it is possible to prevent torque from being accumulated in the outer tubular shaft 70 in a case where this is inserted into a path of the blood vessel bent a plurality of times.

Next, the operator operates the drive unit 92. As a result, the drive shaft 20 rotates, and the cutter 50 and the shaft portion 30 rotate together with the drive shaft 20. As a result, the operator can cut the lesion S by the cutter 50. At that time, especially in the curved portion 78, the inner surface of the outer tubular shaft 70 and the drive shaft 20 rotating at a high speed might come into contact with each other, but since the distal end covering portion 25 made of resin is provided on the distal end portion of the drive shaft 20, friction between the drive shaft 20 and the outer tubular shaft 70 can be controlled to be low.

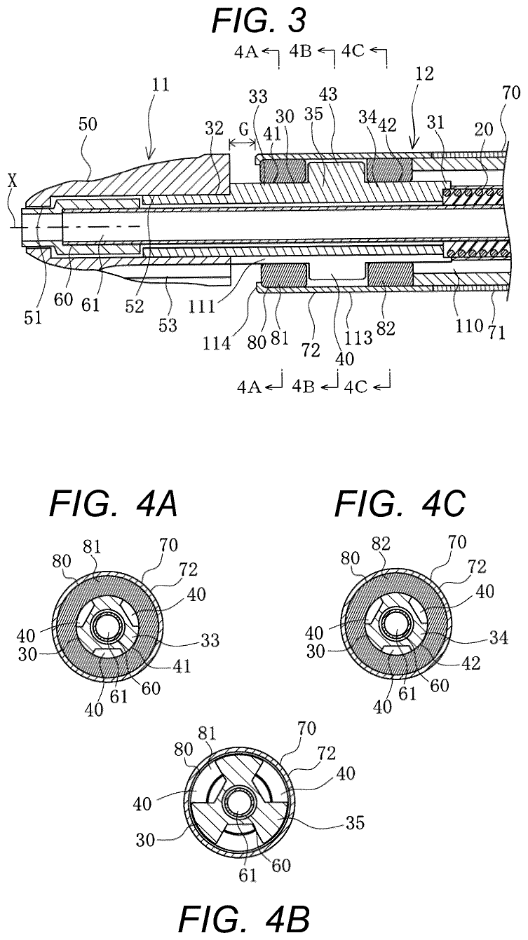

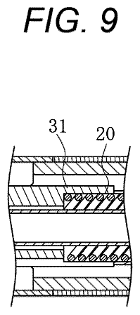

Since the outer tubular shaft 70 includes the S-shaped curved portion 78, the operator can change the direction of the curved portion 78 of the outer tubular shaft 70 and change the position of the cutter 50 by rotating the rotation operation portion 94 in a state of holding the casing 91. As illustrated in FIG. 8, when the cutter 50 is in a position to cut the lesion S, the curved portion 78 abuts the blood vessel wall. When the drive shaft 20 rotates in this state, the distal end portion of the drive shaft 20 is supported by the curved portion 78 abutting the blood vessel wall as a bearing, so that even if the rotation axis deviates, an orbital motion around the axis is less likely to occur. Therefore, as illustrated in FIG. 3, the proximal end coupling portion 31 of the shaft portion 30 that fixes the distal end of the drive shaft 20 is formed to be shallow. In a case where the outer tubular shaft 70 does not include the curved portion 78, there is no portion that supports the drive shaft 20, and when the deviation of the rotation axis occurs, the orbital motion around the axis occurs. As illustrated in FIG. 9, the proximal end coupling portion 31 of the shaft portion 30 is formed to be deep, and the distal end of the drive shaft 20 is housed in the proximal end coupling portion 31 longer in the long axis direction (axial direction), so that it is possible to decrease metal fatigue of the drive shaft 20 caused by the orbital motion and to suppress possibility of breakage. In a case of including the curved portion 78, since the proximal end coupling portion 31 can be formed to be shallow, it is possible to prevent a decrease in flexibility in this portion.

The operator can reciprocate the outer tubular shaft 70 in a longitudinal direction of the blood vessel by moving the entire handle 90 or the outer tubular shaft 70 exposed to the outside of the body. As a result, the operator can cut the lesion S in the longitudinal direction of the blood vessel by the cutter 50.

The additional coil 23 can generate the flow of fluid that conveys an object toward the proximal end side in the intake lumen 110 as the drive shaft 20 rotates. Therefore, the lesion S cut by the cutter 50 becomes debris, and is taken in the intake lumen 110 from the distal end opening. The debris can pass through the cutout 53 of the cutter 50 and efficiently enter the passage 40 communicating with the cutout 53. The debris can enter the passage 40 from the gap G between the cutter 50 and the support tube 72. The passage 40 can be formed spirally around the axis X. As a result, when the drive shaft 20 rotates by the rotating passage 40, the spiral passage 40 that rotates can function as an Archimedean screw (screw pump). As a result, the passage 40 can smoothly convey the object or fluid inside the intake lumen 110 toward the proximal end side.

The debris that enters the passage 40 on the distal end side from the first bearing 81 moves toward the proximal end side inside the first bearing 81 and the second bearing 82. Thereafter, the debris moves from the passage 40 to the outer peripheral surface side of the shaft portion 30 on the proximal end side from the second bearing 82. Thereafter, the debris moves toward the proximal end side in the intake lumen 110 and is discharged through the intake space 95 and the intake port 93. After the cutting of the lesion S and the intake of the debris are completed, the operator stops the operation of the drive unit 92. As a result, the cutting of the lesion S and the discharge of the debris stop. Thereafter, the operator removes the medical device 10 from the blood vessel, and the treatment is completed.

As described above, (1) a medical device 10 according to the present embodiment is a medical device 10 including an elongated drive shaft 20 that rotates around an axis in a longitudinal axis direction (longitudinal axial direction) as a central axis, and an elongated outer tubular shaft 70 disposed outside the drive shaft 20, in which an outer surface of a distal end portion of the drive shaft 20 is covered with a distal end covering portion 25 made of resin. In the medical device 10 configured as described above, since the distal end portion of the drive shaft 20 is covered with a resin, abrasion when the distal end portion of the drive shaft 20 comes into contact with an inner surface of the outer tubular shaft 70 can be decreased, and the drive shaft 20 can be smoothly rotated.
(2) In the medical device 10 according to (1) above, the drive shaft 20 may be a tubular body in which a plurality of wire rods is arranged and spirally coupled around the axis, and at least a part of the distal end covering portion 25 may enter an inside of the drive shaft 20 from between the wire rods. As a result, in the medical device 10, a contact area between the drive shaft 20 and the distal end covering portion 25 increases, and the distal end covering portion 25 can be firmly fixed to the drive shaft 20 so as not to be peeled off.
(3) In the medical device 10 according to (2), the distal end covering portion 25 may include a proximal end and a distal end, and the proximal end may enter more deeply the inside of the drive shaft 20 than the distal end. As a result, the medical device 10 can further prevent peeling on the proximal end of the distal end covering portion 25 while securing flexibility of the distal end portion of the drive shaft 20.
(4) In the medical device according to (3) above, the outer tubular shaft 70 may include a curved portion 78 on a distal end, and the proximal end of the distal end covering portion 25 may be located on a proximal end side from the curved portion 78. As a result, when the drive shaft 20 comes into contact with the outer tubular shaft 70 in the curved portion 78, the abrasion of the drive shaft 20 can be effectively decreased by the distal end covering portion 25.
(5) In the medical device 10 according to (4) above, the curved portion 78 may include a first curved portion 78a and a second curved portion 78b that are separated in a long axis direction (axial direction) and curved in opposite directions to each other. As a result, in the medical device 10, since the drive shaft 20 easily comes into contact with the outer tubular shaft 70, the abrasion of the drive shaft 20 can be effectively decreased by the distal end covering portion 25.
(6) In the medical device 10 according to (3) or (4) above, the distal end covering portion 25 may continuously enter the inside of the drive shaft 20 from a position on a distal end side from the curved portion 78 to a position on the proximal end side from the curved portion 78. As a result, the medical device 10 can prevent peeling of the distal end covering portion 25 even when the drive shaft 20 comes into contact with the inner surface of the outer tubular shaft 70 in the portion of the curved portion 78.
(7) In the medical device 10 according to any one of (2) to (6) above, the distal end covering portion 25 may enter the inside to a depth of more than 0 (zero) μm and 100 μm or less from an imaginary plane connecting surfaces (outer surfaces) farthest from the axis (axial center) of the wire rods. As a result, the medical device 10 can sufficiently secure fixing strength of the distal end covering portion 25 with respect to the drive shaft 20.
(8) In the medical device 10 according to any one of (1) to (7) above, the drive shaft 20 may include an additional coil 23 wound spirally on an outer peripheral surface, and the proximal end position of the distal end covering portion 25 may be on the distal end side from a distal end position of the additional coil 23. As a result, the medical device 10 can prevent an inner cavity of the portion in which the additional coil 23 is provided from being narrowed.

(9) In the medical device 10 according to any one of (1) to (8) above, a protection tube 60 into which a guide wire is inserted may be disposed in the drive shaft 20. As a result, the guide wire can be protected from being damaged by the rotating drive shaft 20.

(10) In the medical device 10 according to (9) above, the protection tube 60 may be disposed so as not to rotate together with the drive shaft 20. This can prevent the guide wire from rotating together when the drive shaft 20 rotates.

The medical device disclosed here is not limited to the embodiment described above, and various modifications can be made by those skilled in the art. The medical device 10 is not limited to the device that cuts and removes the object in the blood vessel, and the medical device can also be applied to other types of devices including the tube made of metal on the distal end such as an ultrasonic catheter, for example.

The detailed description above describes embodiments of a medical device, manner of use and manufacturing method representing examples of the new medical device, manner of use and manufacturing method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A medical device positionable in a biological lumen, the medical device comprising:
an elongated outer tubular shaft configured to be positioned in the biological lumen, the elongated outer tubular shaft having an inner surface surrounding an interior of the elongated outer tubular shaft;
a rotatable elongated drive shaft positioned in the interior of the elongated outer tubular shaft so that a distal portion of the elongated outer tubular shaft surrounds and axially overlaps at least a distal portion of the rotatable elongated drive shaft, the rotatable elongated drive shaft being rotatable relative to the elongated outer tubular shaft, the rotatable elongated drive shaft having an outer surface;
the elongated outer tubular shaft and the rotatable elongated drive shaft being configured so that during rotation of the rotatable elongated drive shaft relative to the elongated outer tubular shaft, the distal portion of the rotatable elongated drive shaft contacts the inner surface of the elongated outer tubular shaft;
a distal end covering portion located at and covering the distal portion of the rotatable elongated drive shaft, the distal end covering portion being made of resin, the distal end covering portion having an outer surface;
the distal end covering portion including a proximal end portion terminating at a proximal end of the distal end covering portion, the distal end covering portion also including a distal end portion terminating at a distal end of the distal end covering portion, the rotatable elongated drive shaft extending proximally beyond the proximal end of the distal end covering; and
the outer surface of the distal end covering portion made of resin reducing friction at a time of the contact of the rotatable elongated drive shaft with the elongated outer tubular shaft during rotation of the rotatable elongated drive shaft relative to the elongated outer tubular shaft.

2. The medical device according to claim 1, further comprising a cutter configured to cut an object located in the biological lumen, the cutter being connected to the rotatable elongated drive shaft so that rotation of the rotatable elongated drive shaft results in rotation of the cutter.

3. The medical device according to claim 1, wherein the rotatable elongated drive shaft comprises one or more wires that are spirally wound so that the rotatable elongated drive shaft includes axially adjacent windings, the resin of the distal end covering portion being positioned between the axially adjacent windings.

4. The medical device according to claim 1, wherein the distal portion of the elongated outer tubular shaft that axially overlaps the distal portion of the rotatable elongated drive shaft is a curved portion of the elongated outer tubular shaft.

5. The medical device according to claim 4, wherein the proximal end of the distal end covering portion is proximal of the curved portion.

6. The medical device according to claim 4, wherein the curved portion of the elongated outer tubular shaft includes a first curved portion and a second curved portion that are separated from one another in an axial direction of the elongated outer tubular shaft and are curved in opposite directions to each other.

7. The medical device according to claim 1, further comprising a coil wound around the outer surface of the rotatable elongated drive shaft, the coil including a distal portion terminating at a distal end of the coil and a proximal portion terminating at a proximal end of the coil, the distal end of the coil being positioned proximal of the proximal end of the distal end covering portion that is made of the resin.

8. The medical device according to claim 1, wherein:
the rotatable elongated drive shaft comprises one or more wires that are spirally wound so that the rotatable elongated drive shaft includes axially adjacent windings;
the resin of the distal end covering portion is positioned between the axially adjacent windings;
the resin of the distal end covering portion extends radially inwardly towards a central axis of the rotatable elongated drive shaft; and
the resin of the proximal end portion of the distal end covering portion extending further radially inwardly towards the central axis of the rotatable elongated drive shaft than the resin of the distal end portion of the distal end covering portion.

9. A medical device comprising:
an elongated drive shaft having a longitudinally extending central axis and being rotatable around the central axis, the drive shaft having an outer surface and including a distal end portion;
an elongated outer tubular shaft disposed outside the drive shaft; and
the outer surface of the distal end of the elongated drive shaft being covered with a distal end covering portion made of resin.

10. The medical device according to claim 9, wherein
the drive shaft is a tubular body comprised of plural wires that are spirally wound around the central axis so the drive shaft includes axially adjacent windings of the wires, and
at least a part of the distal end covering portion being positioned between the axially adjacent windings of the wires.

11. The medical device according to claim 10, wherein the distal end covering portion includes a proximal end and a distal end, and the proximal end of the distal end covering portion enters more deeply toward the central axis of the shaft than the distal end portion of the distal end covering portion.

12. The medical device according to claim 11, wherein
the distal end portion of the outer tubular shaft is a curved portion of the outer tubular shaft, and
the proximal end of the distal end covering portion is located on a proximal end side from the curved portion.

13. The medical device according to claim 12, wherein the curved portion of the outer tubular shaft includes a first curved portion and a second curved portion that are separated from one another in an axial direction of the outer tubular shaft and are curved in opposite directions to each other.

14. The medical device according to claim 12, wherein the distal end covering portion continuously enters the inside of the drive shaft from a position on a distal end side from the curved portion to a position on the proximal end side from the curved portion.

15. The medical device according to claim 10, wherein the distal end covering portion extends radially inwardly towards the central axis of the drive shaft by a depth of more than 0 μm and 100 μm or less from an imaginary plane connecting surfaces farthest from the axis of the wires.

16. The medical device according to claim 9, further comprising a coil wound spirally on an outer peripheral surface of the drive shaft so that axially adjacent windings are axially spaced apart from one another, and
the proximal end of the distal end covering portion is distal of a distal end of the coil.

17. The medical device according to claim 9, wherein a protection tube into which a guide wire is inserted is disposed in the drive shaft.

18. The medical device according to claim 17, wherein the protection tube is disposed so as not to rotate together with the drive shaft.

19. A method comprising:
positioning an elongated outer tubular shaft in a biological lumen, the elongated outer tubular shaft having an inner surface surrounding an interior of the elongated outer tubular shaft in which is positioned an elongated drive shaft so that a distal portion of the elongated outer tubular shaft surrounds and axially overlaps at least a distal portion of the elongated drive shaft, the elongated drive shaft including a distal end covering portion located at and covering the distal portion of the elongated drive shaft, the distal end covering portion being made of resin and having an outer surface, the distal end covering portion including a proximal end portion terminating at a proximal end of the distal end covering portion, the distal end covering portion also including a distal end portion terminating at a distal end of the distal end covering portion, the elongated drive shaft extending proximally beyond the proximal end of the distal end covering portion;
rotating the elongated drive shaft relative to the elongated outer tubular shaft while the elongated outer tubular shaft is positioned in the biological lumen and while the elongated drive shaft is positioned in the interior of the elongated outer tubular shaft;
the rotating of the elongated drive shaft relative to the elongated outer tubular shaft resulting in the outer surface of the distal end covering portion made of the resin to contact the inner surface of the elongated outer tubular shaft; and
the outer surface of the distal end covering portion made of resin reducing friction during the contact of the rotatable elongated drive shaft with the elongated outer tubular shaft while the elongated drive shaft is rotating relative to the elongated outer tubular shaft.

20. The method according to claim 19, wherein the elongated outer tubular shaft includes a curved portion, the distal end covering portion made of the resin axially overlapping the curved portion of the elongated outer tubular shaft.
